(19) United States
(12) Patent Application Publication
(10) Pub. No.: US 2021/0267845 A1
(43) Pub. Date: Sep. 2, 2021

(54) DEVICE COMPRISING AN OBJECT WITH A HEATING AND BIOCOMPATIBLE TIP

(71) Applicant: AXEMOX, Strasbourg (FR)

(72) Inventors: Philippe KORSEC, Strasbourg (FR); Jean-Michel HUBE, Strasbourg (FR); Antoine MATTERN, Strasbourg (FR); Robin MEYER, Strasbourg (FR)

(73) Assignee: AXEMOX, Strasbourg (FR)

(21) Appl. No.: 17/255,075

(22) PCT Filed: Jul. 3, 2019
(86) PCT No.: PCT/FR2019/051644

§ 371 Date: Dec. 22, 2020

(30) Foreign Application Priority Data
Jul. 3, 2018 (FR) 1856116

(51) Int. Cl. A61H 39/08; A61B 17/3211

(52) U.S. Cl. CPC A61H 39/086; A61B 2017/00115; A61B 17/3211

(57) ABSTRACT

A device including an object with a pointed, sharp or heating tip, which is biocompatible and which includes at least one ferromagnetic material, a magnetic field generator suitable for directing the magnetic field of the ferromagnetic material in order to produce an increase in the temperature in the heating tip of the object, and a device for controlling the temperature of the heating tip of the object. Also, the cosmetic use of the device in the treatment of fine lines, dark circles and superficial cellulite. Finally, the use of the device in the treatment of varicose veins and/or in the treatment of syndromes connected to spondylalgia clinical settings and pain connected to these various clinical settings, in particular lumbago or chronic neck pain and their projected pain.

### FIELD OF THE INVENTION

The present invention relates to a device comprising an object with a pointed, sharp, heating tip that is biocompatible that can be used in conventional treatments such as acupuncture or moxibustion, even in any physiopathological clinical setting that indicates moxibustion as a therapeutic means in medicine, and in particular in traditional Chinese medicine.

The present invention can in particular be used in the cosmetic treatment of fine lines, dark circles and superficial cellulite or in the treatment of varicose veins and/or in the treatment of syndromes related to spondylalgia clinical settings and pain related to these various clinical settings, in particular lumbago or chronic neck pain and their projected pain.

### PRIOR ART

Acupuncture or moxibustion are traditional therapeutic techniques linked to traditional Chinese medicine. Acupuncture consists of a stimulation of precise zones of the skin (dermis, epidermis, hypodermis) referred to as “acupuncture points” by means of more or less fine needles. Moxibustion consists of a stimulation by heat of acupuncture points. These techniques can be used for many applications and in particular the treatment or the support for many disorders such as articular disorders, dermatitis, acute or chronic pain, etc.

The use of these conventional techniques sometimes has disadvantages. In particular, in the case of moxibustion, there is, on the one hand, a high risk of burning the skin, and on the other hand, a risk of causing a substantial and uncomfortable release of fumes that can be toxic. In addition, this technique does not make it possible to accurately control the temperature of the heated zone.

Finally, moxibustion cannot be used in a confined space (for example in an office or orbital station) due to the release of fumes unless an aspirating smoke hood is available or moxa that does not release any fumes that can be toxic.

There is therefore a need to change this technique in order to guarantee a secure, reliable and practical use.

Document WO95/20935 has proposed a heating acupuncture needle that comprises an external source of heat and means for heating in the form of a thermal conductor integrated into the needle over the entire length of the latter. Through an application of heat thanks to a heating system on the upper portion of the needle, this results in a transfer by conduction of heat, along the heating system to the tip of the needle and a radiation of this heat through the needle over the entire length of the needle.

The first disadvantage of this technique is that it imposes a needle that comprises several successive layers and is therefore difficult to manufacture. The second disadvantage is, that the various layers realised in order to form the means of heating can, through the repeated application of heat or of electricity, are rapidly deteriorated with the risk of a short-circuit which contraindicates the use. Finally, this technique does not allow for an accurate control of the temperature at the tip of the needle.

Document CN 20276168 describes a moxibustion system where the temperature is controlled by induction. However, this document only relates to “external” moxibustion, i.e. on the surface of the skin. Consequently, there is no way to control the temperature applied inside the body in a precise manner.

Thus, there is a need for a new heating device that makes it possible to improve the conventional techniques.

### SUMMARY

The present invention consists of a device comprising an object which comprises a pointed, sharp, heating tip that is biocompatible, and which comprises at least one ferromagnetic material, a magnetic field generator suitable for directing the magnetic field of the ferromagnetic material in order to produce an increase in the temperature in the heating tip of the object, and a device for controlling the temperature of the heating tip of the object. Said magnetic field generator with the shape of a cylinder which comprises an orifice passed through perpendicularly by said object.

The device according to the invention makes it possible to obtain a precise and secure control of the temperature at the heating tip of the object by means of the device for controlling the temperature. Thus, the temperature of the tip of the object can be perfectly calibrated to a setpoint temperature set by the practitioner. In addition, the device according to the invention is easy to manufacture and to implement.

In an embodiment, said magnetic field generator has the shape of a cylindrical roller, insulating or heating, intended to be placed on the skin, which comprises an orifice passed through perpendicularly by said object.

In an embodiment, said magnetic field generator is formed from a shell made of insulating material.

In an embodiment, said magnetic field generator is formed from a shell made of a conductive material, preferably a shell made of a conductive material.

In an embodiment, said magnetic field generator has a diameter ranging from 10 mm to 100 mm, preferably from 20 mm to 80 mm.

In an embodiment, said magnetic field generator has a thickness ranging from 0.1 mm to 50 mm, preferably from 0.5 mm to 25 mm.

In an embodiment, said orifice of the magnetic field generator has a diameter ranging from 0.1 mm to 10 mm, preferably from 0.1 mm to 5 mm.

In an embodiment, the tip of the object is sharp. In an embodiment, the tip of the object is a scalpel blade.

In an embodiment, the tip of the object is pointed and cylindrical. In an embodiment, the tip of the object is a needle. In an embodiment, the tip of the object is an acupuncture needle.

In an embodiment, the ferromagnetic material is present in the entire object.

In an embodiment, the object is covered at least partially with a layer of insulating material, preferably is covered entirely with a layer of insulating material except for the heating tip.

In an embodiment, the object further comprises a disc, insulating or heating, intended to be placed on the skin which comprises an orifice passed through perpendicularly by the object.

In an embodiment, the magnetic field generator further comprises ferrite arranged in such a way as to direct the magnetic field along the longitudinal axis of the object.

In an embodiment, the device for controlling the temperature of the device of the invention is connected to the magnetic field generator and comprises a) a device for displaying the temperature of the tip of the object, b) means for determining a setpoint temperature, and c) a device for controlling the magnetic field generator so that the temperature of the tip of the object is equal to the setpoint temperature.

In an embodiment, the ferromagnetic material is chosen from steels, preferably chosen from ferritic stainless steels, martensitic stainless steels, and the mixtures of these steels, more preferably chosen from martensitic stainless steel 420, martensitic stainless steel 416, ferritic stainless steel 430 and the mixtures of these steels, and in particular the ferromagnetic material is martensitic stainless steel 420.

In an embodiment, the tip of the object is covered at least partially with a biocompatible material, more preferably the biocompatible material is chosen from silicone, polytetrafluoroethylene (teflon), and a hydrophilic polymer, and more preferably is silicone.

### Definitions

In this invention, the terms hereinbelow are defined as follows:
“Biocompatibility” designates the property of a material that provokes little or no immune response in a given organism when the material is in contact with said organism.
“Projected pain” designates a pain felt at a distance from the causal lesion. Thus, when the projected pain is associated with a pathology, the pain is not localised at the location of the pathology but is due however to this pathology.
“About” placed in front of a number means more or less 10% of the nominal value of this number.
“Ferrite” designates a solid mixture of carbon and of the allotrope a of iron or designates the allotrope a of iron.
“Ferromagnetic” designates the property of a material to form a permanent magnet or to be attracted by a magnet.
“Insulating material” designates a material that makes it possible to thermally or electrically insulate, and more preferably thermally, a contact surface of a thermally or electrically conductive zone, and more preferably thermally.

### BRIEF DESCRIPTION OF THE FIGURES

FIG. 1 is a diagram of a device 1 according to an embodiment of the present invention. In this embodiment, the object that comprises a heating tip 2 is a scalpel.

FIG. 2 is a diagram of a device 1 according to another embodiment of the present invention. In this embodiment, the object that comprises a heating tip 2 is an acupuncture needle.

FIG. 3 is a diagram of a device 1 according to another embodiment of the present invention. In this embodiment, the object that comprises a heating tip 2 is an acupuncture needle. In this embodiment, the magnetic field generator 3 has the shape of a cylindrical roller placed on the skin which comprises an orifice passed through perpendicularly by the object 2.

FIG. 4 is a diagram of a device 1 according to another embodiment of the present invention. In this embodiment, the object that comprises a heating tip 2 is an acupuncture needle and the connection between the object comprising a heating tip and the device for controlling the temperature 4 is done by means of a wireless connection such as for example a Bluetooth or Wi-Fi connection, which makes it possible to add to the device a treatment via suction cup.

FIG. 5 is a diagram of a device 1 according to another embodiment of the present invention. In this embodiment, the device further comprises a disc 6 placed on the skin which comprises an orifice passed through perpendicularly by the object 2.

FIG. 6 is a diagram of a device 1 according to another embodiment of the present invention. In this embodiment, the magnetic field generator 3 further comprises ferrite 7 arranged in such a way as to direct the magnetic field along the longitudinal axis of the object 2.

FIG. 7 is a diagram of a device 1 according to another embodiment of the present invention. In this embodiment, the magnetic field generator 3 has the shape of a cylindrical roller placed on the skin which comprises an orifice passed through perpendicularly by the object 2 and further comprises ferrite 7 arranged in such a way as to direct the magnetic field along the longitudinal axis of the object 2.

FIG. 8 is a horizontal cross-section view of the device 1 of FIG. 6.

FIG. 9 is a diagram of a device 1 according to another embodiment of the present invention. In this embodiment, the object that comprises a heating tip 2 is a scalpel and comprises in the entire object 2 (and therefore also in the heating tip) a ferromagnetic material 9. The object 2 is covered at least partially with a layer of insulating material 10, preferably is covered entirely with a layer of insulating material 10 except for the heating tip.

FIG. 10 is a diagram of the heating tip of the object 2 of the device 1 according to another embodiment of the present invention. In this embodiment, the tip of the object 2 comprises a ferromagnetic material 9 and is covered with a biocompatible material 11.

FIG. 11 is a diagram of the object 2 of the device 1 according to another embodiment of the present invention. In this embodiment, the ferromagnetic material is included in the entire object 2. The object 2 is covered at least partially with a layer of insulating material 10, preferably is covered entirely with a layer of insulating material 10 except for the heating tip. The object 2 is entirely covered with a biocompatible material 11.

FIG. 12 is a diagram of the device for controlling the temperature 4 of the device 1 according to another embodiment of the present invention. In this embodiment, the device for controlling the temperature comprises a device for displaying the temperature of the tip of the object 12 and the setpoint temperature 15, means for determining a setpoint temperature 13, and a switch 14.

### DETAILED DESCRIPTION

The following description shall be understood better when reading the drawings. With the purpose of illustrating the invention, the device is shown in preferred embodiments. It must be understood, however, that the present application is not limited to the arrangements, structures, characteristics, embodiments and precise appearance indicated. The drawings are not to scale and are not intended to limit the scope of the claims to the embodiments shown in these drawings. Consequently, it must be understood that when characteristics mentioned in the claims are followed by references, said references are included solely for the purpose of improving the comprehension of the claims and do not in any way limit the scope of these claims.

Figures

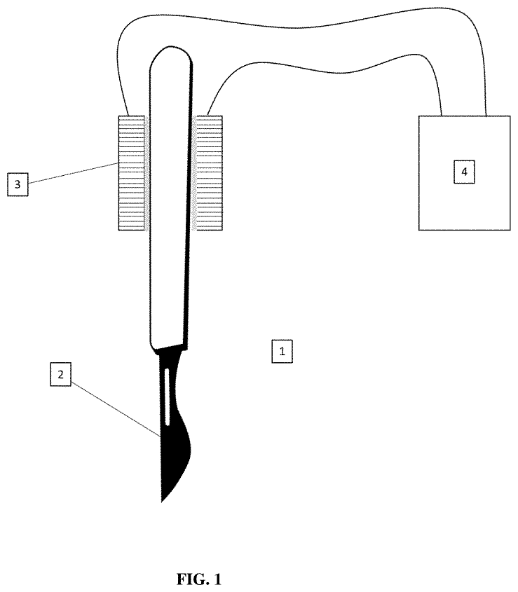

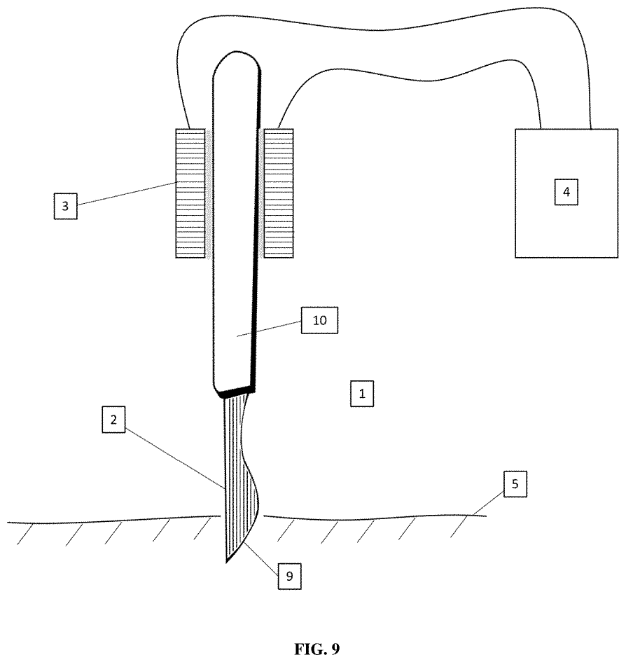

FIGS. 1 and 9 show embodiments according to the invention, wherein the device 1 comprises an object 2 comprising a sharp and heating tip, which is biocompatible, such as a scalpel blade and which comprises at least one ferromagnetic material 9, not shown in FIG. 1. The device also comprises a magnetic field generator 3 suitable for directing the magnetic field of the ferromagnetic material in order to produce an increase in the temperature in the heating tip of the object 2 and a device for controlling the temperature 4 of the heating tip of the object 2. The device for controlling the temperature 4 acts on the magnetic field generator 3 by supplying a current. This current will produce a magnetic field. The magnetic field thus generated will orient the particles of the ferromagnetic material 9 (not shown in FIG. 1) contained in the object 2, more preferably in the entire object 2, and thus produce an increase in the temperature of the entire inside of the object 2. The temperature of the tip of the object 2 can be controlled by means for example of a thermometer or a thermocouple (not shown here), the device for controlling the temperature 4 thus modifying the current provided to the magnetic field generator 3. As shown in FIG. 9, the object 2 is covered at least partially with a layer of insulating material 10, preferably is covered entirely with a layer of insulating material 10 except for the heating tip. This insulating material can be chosen from all existing insulating materials, and in particular can be chosen from plastic materials. This insulating material makes it possible to thermally insulate the rest of the object 2, except for the tip. Thus, only the tip of the object 2 is hot and not the entire object 2. The skin and the organs that can be in contact with the rest of the object 2 are not heated.

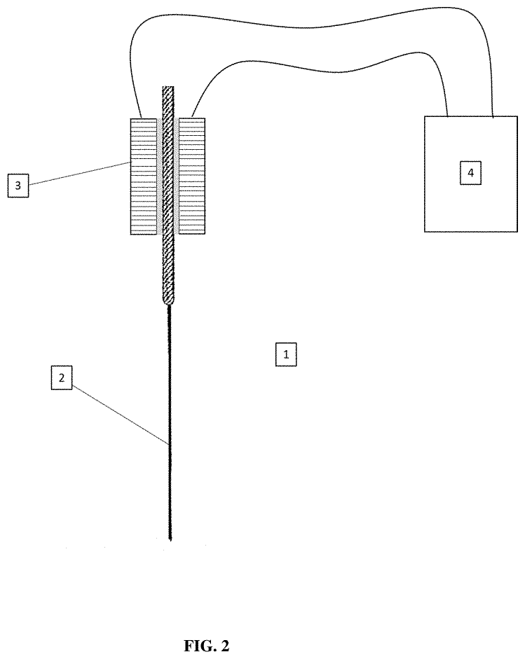

FIGS. 2, 3, 4, 5, 6, 7 and 8 show embodiments according to the invention, wherein the device 1 comprises an object 2 that comprises a pointed heating tip that is biocompatible, such as an acupuncture needle and which comprises at least one ferromagnetic material 9, not shown in these figures. In these embodiments, the device also comprises a magnetic field generator 3 suitable for directing the magnetic field of the ferromagnetic material in order to produce an increase in the temperature in the heating tip of the object 2 and a device for controlling the temperature 4 of the heating tip of the object 2. In an embodiment, the magnetic field generator 3 is not fastened to the object 2. The device for controlling the temperature 4 acts on the magnetic field generator 3 by supplying a current. This current will produce a magnetic field. The magnetic field thus generated will orient the particles of the ferromagnetic material 9 (not shown here) contained in the object 2, more preferably in the entire object 2, and thus produce an increase in the temperature of the entire inside of the object 2. The temperature of the tip of the object 2 can be controlled by means for example of a thermometer or a thermocouple (not shown here), the device for controlling the temperature 4 thus modifying the courant provided to the magnetic field generator 3.

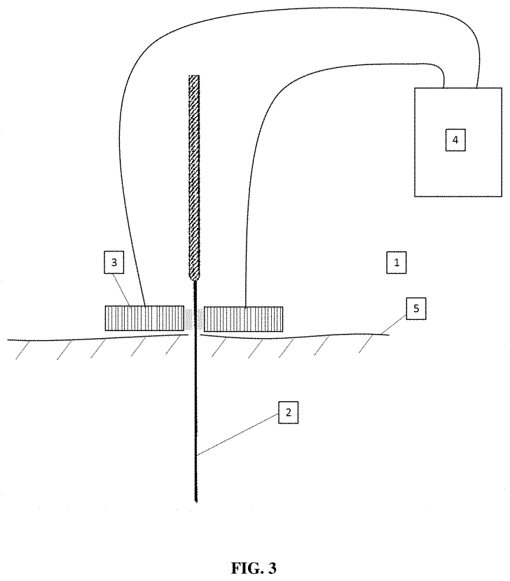

FIG. 3 shows an embodiment according to the invention, wherein the magnetic field generator 3 of the device 1 has the shape of a cylindrical roller, intended to be placed on the skin 5 which comprises an orifice passed through perpendicularly by the object 2. In an embodiment, said roller is insulating, i.e. it is generally formed from a shell made of insulating material, such as a shell made of plastic material, glass or aluminium. In another embodiment, said roller is heating, i.e. it is generally formed from a shell made of conductive material, such as a conductive metal. In this embodiment, the conductive material that forms the shell of said roller is preferably the same as that of the heating tip of the object 2. When said roller is heating, it can be heated thanks to the generated magnetic field which will orientate the particles of the ferromagnetic material 9 (not shown here) contained in the shell of said roller. The use of said roller formed from a shell made of insulating material makes it possible to not heat the skin and to concentrate the heating only on the heating tip of the object 2, for example for localised treatments. The use of said roller formed from a shell made of conductive material makes it possible to heat the skin, for example for non-localised treatments.

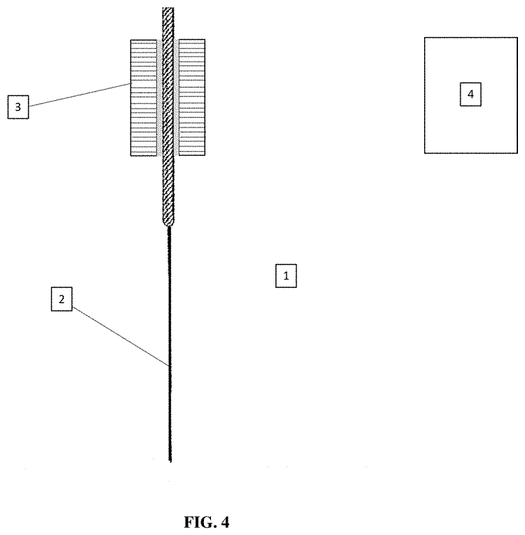

FIG. 4 shows an embodiment according to the invention, wherein the object 2 and the magnetic field generator 3 are connected to the device for controlling the temperature 4 by means of a wireless connection. This wireless connection can be for example a Bluetooth connection or a Wi-Fi connection which makes it possible to add to the device a treatment via suction cup.

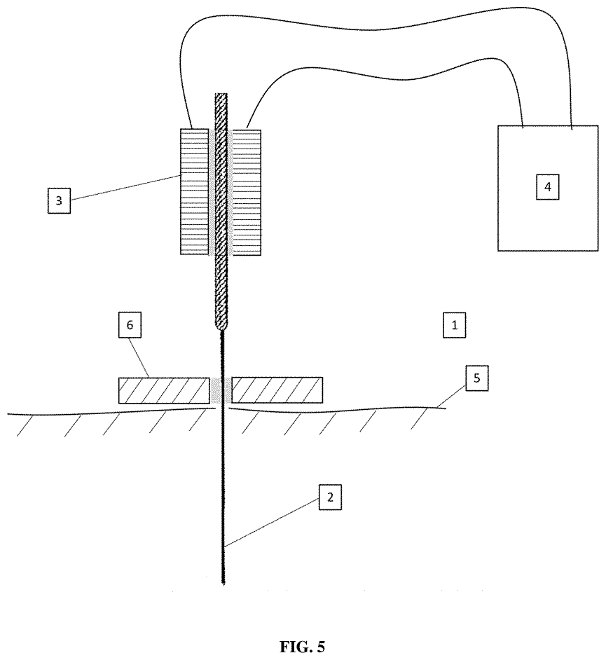

FIG. 5 shows an embodiment according to the invention, wherein the device 1 further comprises a disc 6, insulating or heating, intended to be placed on the skin 5 which comprises an orifice passed through perpendicularly by the object 2. In an embodiment, the disc 6 is insulating, i.e. it is generally formed from an insulating material, such as a plastic material or glass. In another embodiment, the disc 6 is heating, i.e. it is generally formed from a conductive material, such as a conductive metal. In this embodiment, the conductive material that forms the disc is preferably the same as that of the heating tip of the object 2. When the disc 6 is heating, it can be heated in two ways: either independently of the heating tip of the object 2 for example by a heating system or by the object 2 by heat transfer via conduction. The additional use of the insulating disc 6 makes it possible to not heat the skin and to concentrate the heating only on the heating tip of the object 2, for example for localised treatments. The additional use of the heating disc 6 makes it possible to heat the skin, for example for non-localised treatments.

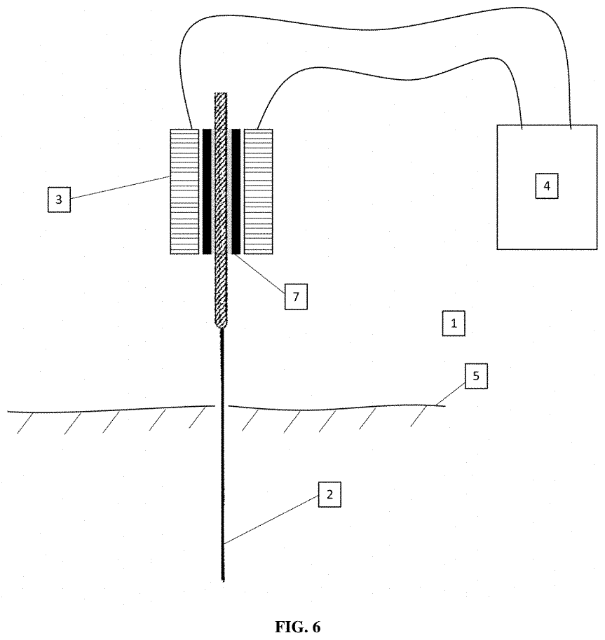

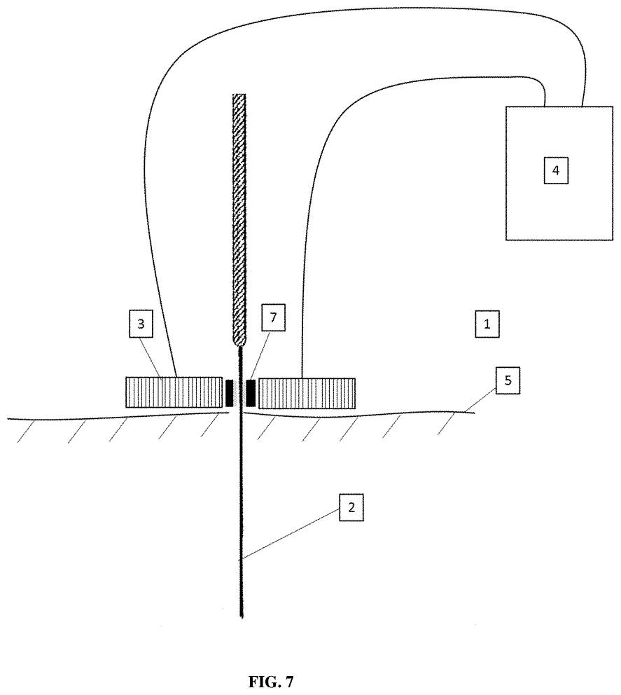

FIGS. 6 and 7 show an embodiment according to the invention, wherein the magnetic field generator 3 of the device 1 further comprises ferrite 7 arranged in such a way as to direct the magnetic field along the longitudinal axis of the object 2. The use of the ferrite 7 has the advantage of directing the magnetic field produced by the magnetic field generator 3 to the tip of the heating object 2. This makes it possible in particular to control, in a fine manner, the temperature reached by the tip of the heating object 2.

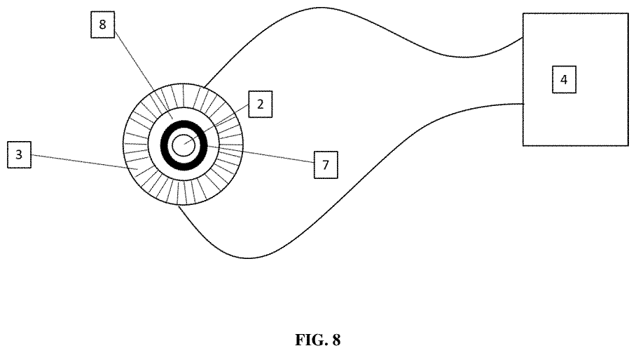

FIG. 8 shows as a horizontal cross-section the embodiment such as described in FIGS. 5 and 7. The ferrite 7 has the shape of a cylinder and is arranged around the object 2. The magnetic field generator 3 also has the shape of a cylinder and is arranged around the ferrite 7. The support 8, for example made of plastic material, is used to link the magnetic field generator 3 to the ferrite 7, with the whole being linked to the object 2, with this link not being shown here.

FIG. 9 shows an embodiment according to the invention, wherein the ferromagnetic material 9 is located in any object 2. The object 2 is covered at least partially with a layer of insulating material 10, preferably is covered entirely with a layer of insulating material 10 except for the heating tip. This insulating material can be chosen from all existing insulating materials, and in particular can be chosen from plastic materials. This insulating material makes it possible to thermally insulate the rest of the object 2, except for the tip. Thus, only the tip of the object 2 is hot and not the entire object 2. The skin and the organs that can be in contact with the rest of the object 2 are not heated.

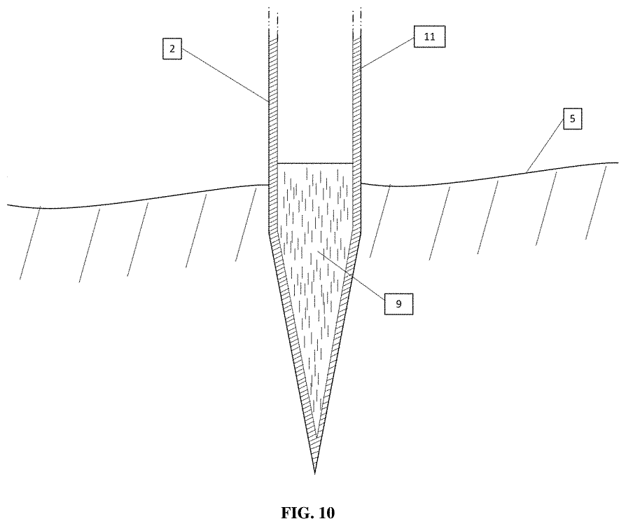

FIG. 10 shows an enlargement of an embodiment according to the invention, wherein the device 1 comprising an object 2 that comprises a pointed heating tip that is biocompatible, such as an acupuncture needle and which comprises at least one ferromagnetic material 9. The heating and pointed tip is surrounded by a biocompatible material 11 such as silicone, polytetrafluoroethylene (teflon), a hydrophilic polymer, and preferably is silicone.

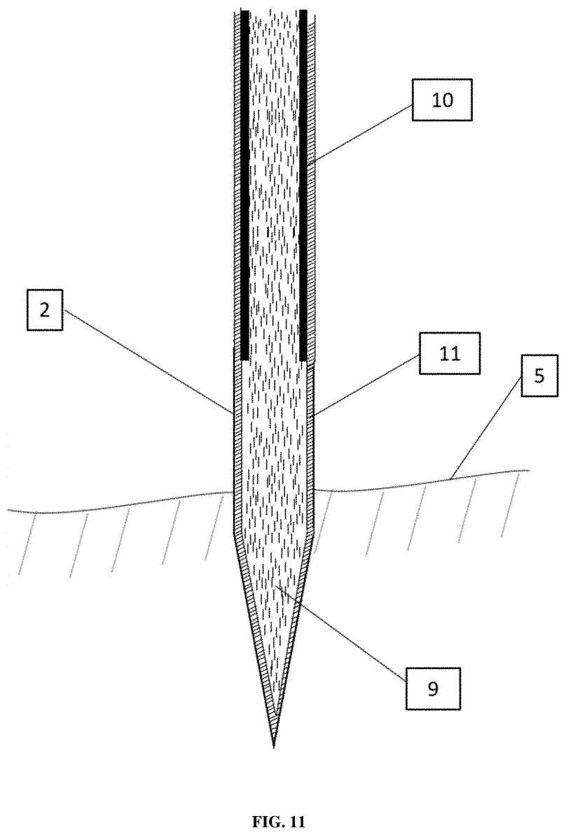

FIG. 11 shows an enlargement of an embodiment according to the invention, wherein the device 1 comprising an object 2 that comprises a pointed heating tip that is biocompatible, such as an acupuncture needle and which comprises at least one ferromagnetic material 9. The ferromagnetic material 9 is present in the entire object 2 The object 2 is covered at least partially with a layer of insulating material 10, preferably is covered entirely with a layer of insulating material 10 except for the heating tip. This insulating material can be chosen from all existing insulating materials, and in particular can be chosen from plastic materials. The object 2 is entirely covered with a biocompatible material 11 such as silicone, polytetrafluoroethylene (teflon), a hydrophilic polymer, and preferably the biocompatible material is silicone.

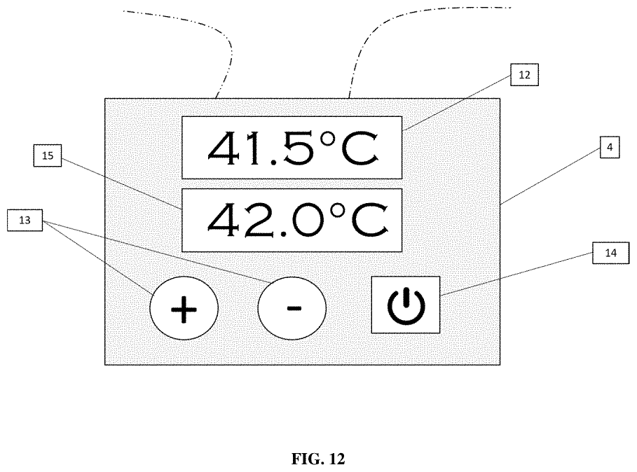

FIG. 12 shows a device for controlling the temperature 4 of the device 1 of an embodiment according to the invention. In this embodiment, the device for controlling the temperature comprises a device for displaying the temperature of the tip of the object 12 and the setpoint temperature 15, means 13 for determining a setpoint temperature, such as for example a +button and a −button, and a switch 14. In this embodiment, the user can set a setpoint temperature 14 by using means 13. Once this setpoint temperature 15 is determined, the temperature of the tip of the object, shown by the device for displaying 12, will change until the setpoint temperature 15 is reached. The temperature of the tip of the object 2 can be measured for example by means of a thermometer or a thermocouple, not shown here, connected to the device for controlling the temperature 4. For safety reasons, the user can constantly maintain control on the rising temperature of the tip of the object 2 by means of the switch 14 which makes it possible to completely interrupt the heating of the tip of the object 2. This switch 14 can also be used to stop at the end of the use the device 1 according to the invention.

The embodiments shown in the rest of the description apply to all of the description and in particular to all of the embodiments described in the figures of the application.

Heating Tip

In an embodiment of the invention, the heating tip of the object 2 is sharp, i.e. it has an elongated shape that has a side that is worked in such a way as to cut material, in particular the skin. In an embodiment, the heating tip is a scalpel blade or lancet. In an embodiment, the object 2 is a scalpel or lancet.

When the heating tip of the object 2 is sharp, in an embodiment, the heating tip can measure from 1 to 40 mm, preferably from 1 to 20 mm, more preferably from 3 to 10 mm.

When the heating tip of the object 2 is sharp, in an embodiment, the thickness of the tip of the object 2 varies by about 0.10 mm to 2.0 mm.

In another embodiment of the invention, the heating tip of the object 2 is pointed, i.e. it has an elongated and cylindrical shape and which is sufficiently fine to be able to pierce material in particular the skin. In an embodiment, the heating tip is a needle, more preferably an acupuncture needle.

When the heating tip of the object 2 is pointed, in an embodiment, the heating tip can measure from 1 to 40 mm, preferably from 1 to 20 mm, more preferably from 3 to 10 mm.

When the heating tip of the object 2 is pointed, in an embodiment, the diameter of the tip of the object 2 varies by about 0.15 mm to about 3.3 mm.

The heating tip of the object 2 of the device 1 according to the invention comprises at least one ferromagnetic material. In an embodiment, the ferromagnetic material is present only in the heating tip of the object 2. In another embodiment, the ferromagnetic material is present in the entire object 2.

Ferromagnetic Material

In an embodiment, the ferromagnetic material 9 is chosen from steels, preferably chosen from ferritic stainless steels, martensitic stainless steels, and the mixtures of these steels,

In an embodiment, the ferromagnetic material 9 is chosen from martensitic stainless steel 420, martensitic stainless steel 416, ferritic stainless steel 430, and the mixtures of these steels.

In an embodiment, the ferromagnetic material 9 is martensitic stainless steel 420. This material has the advantage of being both ferromagnetic and biocompatible.

Biocompatible Material

In an embodiment, the heating tip of the object 2 is covered with a biocompatible material.

In an embodiment, the object 2 is entirely covered with a biocompatible material.

In an embodiment, the biocompatible material is martensitic stainless steel 420. In an embodiment, the ferromagnetic material and the biocompatible material are a single and same compound, martensitic stainless steel 420.

In another embodiment, the biocompatible material is silicone, polytetrafluoroethylene (teflon) or a hydrophilic polymer, and preferably is silicone.

In an embodiment, the ferromagnetic material is martensitic stainless steel 416 and the biocompatible material is silicone.

In an embodiment, the ferromagnetic material is ferritic stainless steel 430 and the biocompatible material is silicone.

In an embodiment, the heating tip of the object 2 is covered with a layer of biocompatible material, for example silicone, with a thickness ranging from 1 μm to 25 μm, preferably ranging from 1 μm to 10 μm.

Insulating Material

In an embodiment, the object 2 is covered at least partially with a layer of insulating material 10. In an embodiment, the object 2 is entirely covered with a layer of insulating material 10 except for the heating tip. In an embodiment, the object 2 is entirely covered with a layer of insulating material 10 except for a zone in contact with a heating disc such as described hereinafter, when the latter is used. In an embodiment, the object 2 is entirely covered with a layer of insulating material 10 except for the heating tip and a zone in contact with a heating disc such as described hereinafter, when the latter is used.

This insulating material can be chosen from all existing insulating materials, and in particular can be chosen from plastic materials.

In an embodiment, the object 2 is entirely covered with a layer of insulating material 10 except for the heating tip forming a first layer and the object 2 is entirely covered with a biocompatible material forming a second layer. Thus, on the heating tip, there is a single layer, the biocompatible material, while for the rest of the object 2, there are two layers, the insulating material and the biocompatible material, with the understanding that the biocompatible material is outside of the object 2 (the portion in contact with the skin or the organs).

In an embodiment, the object 2 is entirely covered with a layer of insulating material 10 except for the heating tip and a zone in contact with a heating disc such as described hereinafter, when the latter is used, forming a first layer and the object 2 is entirely covered with a biocompatible material forming a second layer. Thus, on the heating tip and on the zone in contact with a heating disc such as described hereinafter, there is a single layer, the biocompatible material, while for the rest of the object 2, there are two layers, the insulating material and the biocompatible material, with the understanding that the biocompatible material is outside of the object 2 (the portion in contact with the skin or the organs).

Magnetic Field Generator

In an embodiment, the magnetic field generator 3 is arranged around the object 2. In an embodiment, the magnetic field generator 3 in the form of a hollow cylinder suitable for surrounding the object 2. In an embodiment, the magnetic field generator 3 has the shape of a cylinder which comprises an orifice passed through perpendicularly by the object 2. In an embodiment, the magnetic field generator 3 has the shape of a cylindrical roller intended to be placed on the skin 5 which comprises an orifice passed through perpendicularly by the object 2.

In an embodiment, the magnetic field generator 3 is insulating, i.e. it is formed from a shell made of insulating material. In an embodiment, the magnetic field generator 3 is formed from a shell made of plastic material. In an embodiment, the magnetic field generator 3 is formed from a shell made of glass. In an embodiment, the magnetic field generator 3 is formed from a shell made of aluminium.

When the magnetic field generator 3 has the shape of a cylindrical roller intended to be placed on the skin 5, in an embodiment, said roller is heating, i.e. it is generally formed from a shell made of conductive material. In an embodiment, said roller is formed from a shell made of a conductive metal. In an embodiment, the conductive metal is the same as that of the heating tip of the object 2. In an embodiment, the conductive metal is the same as that of the object 2.

In an embodiment, the magnetic field generator 3 is not fastened to the object 2.

In an embodiment, the magnetic field generator 3 has a diameter ranging from 10 mm to 100 mm, preferably from 20 mm to 80 mm. In an embodiment, the magnetic field generator 3 has a thickness ranging from 0.1 mm to 50 mm, preferably from 0.5 mm to 25 mm. In an embodiment, the orifice of the magnetic field generator 3 has a diameter ranging from 0.1 mm to 10 mm, preferably from 0.1 mm to 5 mm.

In an embodiment, the magnetic field generator 3 comprises a conductive coil passed through by an alternating current.

In an embodiment, the coil has a diameter ranging from 5 mm to 50 mm, preferably ranging from 5 mm to 30 mm. In an embodiment, the coil has a length ranging from 10 mm to 100 mm, preferably ranging from 20 mm to 50 mm. In an embodiment, the coil comprises a number of turns ranging from 50 to 1000, preferably ranging from 100 to 1000.

In an embodiment, the coil is formed from copper wire.

In an embodiment, the magnetic field generator 3 produces a magnetic field of an intensity ranging from 0.01 to 5 T, preferably from 0.1 to 1 T.

In an embodiment, the magnetic field generator 3 comprises several coils such as described hereinabove. In an embodiment, the magnetic field generator 3 comprises two coils such as described hereinabove. In an embodiment, the magnetic field generator 3 comprises three coils such as described hereinabove. In an embodiment, the magnetic field generator 3 comprises four coils such as described hereinabove. These various coils can be controlled separately by the device for controlling the temperature of the device of the invention which makes it possible, on the one hand, to further refine the control of the temperature, and on the other hand to cover a large range of temperatures. Thus, these various coils can be activated separately according to need.

In an embodiment, the magnetic field generator can be activated periodically. Thus, the magnetic field generator makes it possible to produce periods of heating and of cooling of the tip of the object 2, for example according to a cyclical program or according to a program established beforehand. In an embodiment, the magnetic field generator is provided with a frequency modulator.

In an embodiment, the device for controlling the temperature of the device of the invention is connected to the magnetic field generator and controls the magnetic field generator according to the temperature of the tip of the object and of a setpoint temperature.

In an embodiment, the device for controlling the temperature of the device of the invention is connected to the magnetic field generator and comprises a) a device for displaying the temperature of the tip of the object, b) means for determining a setpoint temperature, and c) a device for controlling the magnetic field generator so that the temperature of the tip of the object is equal to the setpoint temperature.

In another embodiment, the device for controlling the temperature of the device of the invention is connected to the magnetic field generator and comprises a) a device for displaying the temperature of the tip of the object and another device for displaying the temperature on the heating disc such as described hereinafter, when the latter is used, b) means for determining a setpoint temperature, and c) a device for controlling the magnetic field generator so that the temperature of the tip of the object and the temperature on the heating disc such as described hereinafter, when the latter is used are equal to the setpoint temperature.

Ferrite

In an embodiment, the magnetic field generator 3 further comprises ferrite 7 arranged in such a way as to direct the magnetic field along the longitudinal axis of the object 2.

In an embodiment, the ferrite has the form of a hollow cylinder suitable for surrounding the object 2.

Disc

In an embodiment, the device 1 further comprises a disc 6, insulating or heating, intended to be placed on the skin 5 which comprises an orifice passed through perpendicularly by the object 2.

In an embodiment, the disc 6 is insulating, i.e. it is formed from an insulating material. In an embodiment, the disc 6 is formed from a plastic material. In an embodiment, the disc 6 is formed from glass.

In another embodiment the disc 6 is heating, i.e. it is generally formed from a conductive material. In an embodiment, the disc 6 is formed from a conductive metal. In an embodiment, the conductive metal is the same as that of the heating tip of the object 2. In an embodiment, the conductive metal is the same as that of the object 2.

In an embodiment, the disc 6 has a diameter ranging from 10 mm to 100 mm, preferably from 20 mm to 80 mm. In an embodiment, the disc 6 has a thickness ranging from 0.1 mm to 5 mm, preferably from 0.5 mm to 2 mm. In an embodiment, the orifice of the disc 6 has a diameter ranging from 0.1 mm to 10 mm, preferably from 0.1 mm to 5 mm

Cosmetic Use

In an embodiment, the device 1 of the invention is used in the treatment of fine lines, dark circles and superficial cellulite. In this embodiment, the device for controlling the temperature of the device of the invention is configured so that the temperature of the tip of the object 2 is generally between 40° C. and 50° C., preferably between 40° C. and 42° C., and more preferably is equal to about 41.5° C.

Therapeutic Use

In an embodiment, the device 1 of the invention is used in the treatment of varicose veins. In this embodiment, the device for controlling the temperature of the device of the invention is configured so that the temperature of the tip of the object 2 is generally between 40° C. and 50° C., preferably between 40° C. and 42° C., and more preferably is equal to about 41.5° C.

In another embodiment, the device 1 of the invention is used in the treatment of syndromes related to spondylalgia clinical settings and pain related to these various clinical settings, in particular lumbago or chronic neck pain and their projected pain. In this embodiment, the device for controlling the temperature of the device of the invention is configured so that the temperature of the tip of the object 2 is generally between 75° C. and 95° C., more preferably is between 80° C. and 90° C.

In an embodiment, the device 1 of the invention is used as a replacement for radiofrequency techniques (or nucleoplasty). In an embodiment, the device 1 of the invention is used as a replacement for radiofrequency techniques of the leg. In this embodiment, the device for controlling the temperature of the device of the invention is configured so that the temperature of the tip of the object 2 is between 100° C. and 200° C., and more preferably is between 100° C. and 130° C.

In an embodiment, the device 1 of the invention is used as an alternative to the thermal treatments such as oxygen-ozone nucleolysis or the use of a laser for percutaneous discectomy with a Holmium-Yag laser. These two techniques are for example described in the thesis of Daniel SPAETER defended on 21 Jun. 2004 entitled “Treatment of sciatica via the percutaneous route: interest of nucleoplasty via radiofrequency, concerning 15 cases” with the jury comprised of: Mr A Gangi, Mr J. L. Dietemann, Mr J. P. Steib, Mr. X. Buy and Mr. P. Laurent.

In an embodiment, the device 1 of the invention can be used in the treatment of certain cancers, in particular on solid tumours of cancers.

In an embodiment, the device 1 of the invention is used as an alternative to an electric lancet. In this embodiment, the tip of the heating object is generally sharp.

Although various embodiments have been described and shown, the detailed description must not be considered as being limited to the latter. Various modifications can be made to the embodiments by those skilled in the art without leaving the true spirit and the scope of the disclosure such as defined by the claims.

### REFERENCES

1—Device
2—Object comprising a heating tip
3—Magnetic field generator
4—Device for controlling the temperature of the heating tip of the object
5—Skin
6—Disc
7—Ferrite
8—Support
9—Ferromagnetic material
10—Insulating material
11—Biocompatible material
12—Device for displaying the temperature of the tip of the object
13—Means for determining a setpoint temperature
14—Switch
15—Device for displaying the setpoint temperature

1-15. (canceled)

16. A device comprising:
an object comprising a pointed, sharp or heated tip, that is biocompatible, and which comprises at least one ferromagnetic material,
a magnetic field generator suitable for directing the magnetic field of the ferromagnetic material in order to produce an increase in the temperature in the heating tip of the object, and
a device for controlling the temperature of the heating tip of the object wherein said magnetic field generator with the shape of a cylinder which comprises an orifice passed through perpendicularly by said object.

17. The device according to claim 16, wherein the magnetic field generator with the shape of a hollow cylindrical roller, intended to be placed on the skin, which comprises an orifice passed through perpendicularly by said object.

18. The device according to claim 16, wherein the magnetic field generator is formed from a shell made of insulating material.

19. The device according to claim 17, wherein the magnetic field generator is formed from a shell made of a conductive material, preferably a shell made of a conductive material.

20. The device according to claim 16, wherein the magnetic field generator has a diameter ranging from 10 mm to 100 mm, preferably from 20 mm to 80 mm and a thickness ranging from 0.1 mm to 50 mm, preferably from 0.5 mm to 25 mm.

21. The device according to claim 16, wherein said orifice of the magnetic field generator has a diameter ranging from 0.1 mm to 10 mm, preferably from 0.1 mm to 5 mm.

22. The device according to claim 16, wherein said tip of the object is sharp, and preferably is a scalpel blade.

23. The device according to claim 16, wherein said tip of the object is pointed and cylindrical, preferably is a needle, and in particular an acupuncture needle.

24. The device according to claim 16, wherein the ferromagnetic material is present in the entire object.

25. The device according to claim 16, wherein the object is covered at least partially with a layer of insulating material, preferably is covered entirely with a layer of insulating material except for the heating tip.

26. The device according to claim 16, further comprising a disc, insulating or heating, intended to be placed on the skin which comprises an orifice passed through perpendicularly by said object.

27. The device according to claim 16, wherein said magnetic field generator further comprises ferrite arranged in such a way as to direct the magnetic field along the longitudinal axis of said object.

28. The device according to claim 16, wherein said device for controlling the temperature is connected to the magnetic field generator and comprises:
a) a device for displaying the temperature of the tip of the object,
b) means for determining a setpoint temperature, and
c) a device for controlling the magnetic field generator so that the temperature of the tip of the object is equal to the setpoint temperature.

29. The device according to claim 16, wherein said ferromagnetic material is chosen from steels, preferably chosen from ferritic stainless steels, martensitic stainless steels, and the mixtures of these steels, more preferably chosen from martensitic stainless steel 420, martensitic stainless steel 416, ferritic stainless steel 430 and the mixtures of these steels, and in particular the ferromagnetic material is martensitic stainless steel 420.

30. The device according to claim 16, wherein said tip of the object is covered at least partially with a biocompatible material, preferably the biocompatible material is chosen from silicone, polytetrafluoroethylene (teflon) and a hydrophilic polymer and preferably is silicone.
